(19) United States
(12) Patent Application Publication
(10) Pub. No.: US 2003/0108592 A1
(43) Pub. Date: Jun. 12, 2003

(54) Method of producing a nicotine composition

(76) Inventor: Marshall Anlauf Thompson, Camarillo, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 10/216,023

(22) Filed: Aug. 9, 2002

Related U.S. Application Data
(63) Continuation-in-part of application No. 10/015,101, filed on Dec. 10, 2001, now abandoned.

(51) Int. Cl. A61K 35/78; A61K 9/48; A61K 31/4439

(52) U.S. Cl. 424/439; 424/751; 514/343

(57) ABSTRACT

A method of producing a composition having a precise amount of nicotine appropriate for a single use by a single individual involves diluting a large quantity of nicotine into one or more successive intermediate solutions, a last of which constitutes a final solution. The final solution is then apportioned, so that each portion contains a precise quantity of nicotine appropriate for consumption in a single use by a single person. The portions may be introduced into beverages and consumed as a cigarette substitute by individuals attempting to quit smoking. This method of nicotine dilution enables accurate and inexpensive production of nicotine compositions having single-serving amounts of nicotine without requiring expensive, precision equipment for measurements.

### RELATED APPLICATION

This is a continuation-in-part of U.S. patent application Ser. No. 10/015,101, filed Dec. 10, 2001.

### BACKGROUND OF THE INVENTION

The present invention relates generally to nicotine beverages that serve as cigarette substitutes for individuals attempting to quit smoking. More particularly, the present invention relates to a method for producing a consumable composition having a precise, single-serving quantity of nicotine using conventional beverage production equipment.

When the cigarette-rolling machine was patented in 1881, it enabled a relatively minor product of the time, the cigarette, to become an extremely popular product. It increased cigarette production by 5000 percent and cut the price of an individual cigarette in half. However, the increase in the cigarette's popularity also brought with it a host of health-related problems.

Today, many people attempting to quit smoking have turned to substitute products containing nicotine. Doctors have long recommended that individuals attempting to quit smoking should consume large amounts of fluids. Fluid consumption in itself is necessary for individual health. Additionally, persons who stop smoking often misinterpret cravings for a cigarette as hunger, which frequently results in weight gain. Drinking fluids helps suppress the cravings for a cigarette and helps curb weight gain. Also, drinking fluids incorporates the same hand-to-mouth motion associated with smoking, which may be psychologically helpful to persons attempting to quit smoking.

In light of the foregoing, an especially effective cigarette alternative is the nicotine beverage. In addition to the advantages listed above, it also has a built-in protection against nicotine abuse, because the nicotine concentration in a beverage can be adjusted so that an individual will reach his or her fluid intake limit before consuming a dangerous amount of nicotine.

As noted above, the cigarette-rolling machine enabled a huge increase in cigarette production as well as a substantial decrease in production cost, thereby allowing the cigarette to become an extremely popular product. The object of the present invention is to have a similar effect on the nicotine beverage market through a method which allows for increased beverage production as well as decreased production costs. This will result in nicotine beverages becoming more available, less expensive, and more popular with consumers.

It is well known that nicotine is a potent substance. Very small amounts of nicotine can produce noticeable effects in adult humans. In fact, the amount of nicotine appropriate for human consumption in a single use, i.e. the amount required to effectively serve as a single cigarette substitute, is too small to be accurately measured in a conventional beverage production setting. The equipment is not sufficiently precise and any error in measurement could mean lethal results for consumers of the beverage.

It is possible to measure nicotine into single-serving amounts by utilizing precision equipment. However, such equipment is expensive and normally unavailable in conventional beverage production settings. In order to be an attractive substitute for cigarettes, a nicotine beverage must be relatively inexpensive. The use of precision measuring equipment will result in higher-priced nicotine beverages, which will be less attractive to individuals attempting to quit smoking.

Accordingly, there is a need for a method of inexpensively producing a composition containing a precise amount of nicotine appropriate for a single use by a single individual. Such a method should not require the use of high-precision measuring equipment, but should instead utilize equipment already found in conventional beverage production settings. Additionally, to increase marketability, such a method should be capable of yielding a variety of compositions, including liquids, solids, tablets, pills, and powders. The present invention fulfills these needs and provides other related advantages.

### SUMMARY OF THE INVENTION

The present invention resides in a method for producing a composition containing a precise amount of nicotine appropriate for a single use by a single individual. The method utilizes equipment normally found in conventional beverage production settings, and functions by diluting nicotine into one or more successive intermediate solutions before yielding the final composition to be mixed into a single-serving beverage. This method of successively diluting the nicotine eliminates the need for expensive, high-precision measurement equipment. As a result, nicotine beverages will be less expensive to produce and will have the potential to reach a larger market.

More particularly, the present invention comprises a method of producing a consumable nicotine composition, including the steps of measuring a large quantity of nicotine, diluting the large quantity of nicotine into one or more successive intermediate solutions, the last of which constitutes a final solution, a portion in the final solution so that each portion contains a precise quantity of nicotine appropriate for consumption in a single use by a single person, and introducing each portion into its own single-serving beverage.

In this regard, each successive intermediate solution has a lower nicotine concentration than that of the solution immediately preceding it. Moreover, a nicotine concentration of between 0.0001% and 0.1% by volume is attained in the beverages after addition of each portion thereto. The primary component may be water, carbonated water or a natural juice, and the final solution may be liquid or non-liquid.

Additional features and advantages of the present invention will become apparent from the following more detailed description.

### DETAILED DESCRIPTION OF THE INVENTION

The present invention is a method for producing a composition containing a precise amount of nicotine appropriate for a single use by a single individual. The nicotine composition may be introduced into a single-serving beverage container in a production setting, or it may be packaged in a single-use dispenser and sold to consumers. Regardless of how the composition is made available, the method of producing the composition remains the same and is the subject of the present invention.

The method involves diluting a known, large quantity of nicotine into successive intermediate solutions, whereby the nicotine concentration is progressively reduced. For example, it is known that a cigarette contains roughly 1 to 4 mg of nicotine. Therefore, a nicotine beverage ought to contain an equivalent amount. Since 1 mg of nicotine is too small to be accurately measured in a conventional beverage production setting, the method of the present invention may be employed, for example, as follows: First, a relatively large quantity of nicotine (one easily measurable in a beverage production setting) is mixed into a measured quantity of water, or other solvent, to produce a first intermediate solution. The nicotine concentration in this first intermediate solution may still be too high for human consumption. In that case, the first intermediate solution is then divided into a number of equal portions. Each portion is mixed into its own, separate quantity of water, yielding a number of second intermediate solutions. The nicotine concentrations in each of these second intermediate solutions should be equal to one another, and substantially lower than the nicotine concentration in the first intermediate solution.

This process of diluting successive intermediate solutions will eventually yield a final solution having the precise nicotine concentration desired. The number of dilutions and intermediate solutions required will depend on the desired final nicotine concentration, the size of the available containers for dilution and mixing, and the precision of the available measuring equipment. It is quite possible that the first intermediate solution will contain the desired nicotine concentration.

The final nicotine composition may be introduced into single-serving beverage containers, such as cans or bottles, in a production setting, or it may be packaged in single-use dispensers to be mixed into beverages later. In the latter case, the final nicotine composition may even be evaporated to yield the single-serving quantity of nicotine contained therein, which may then be mixed with a water-soluble powder filler and sealed in a packet. Such a packet would contain an appropriate amount of nicotine for a single serving and could be opened and its contents mixed into any beverage of choice.

It should be noted that while water is specifically mentioned in describing the dilution process, the method of the present invention may employ a wide range of substances as a solvent. It should also be noted that the method of the present invention may be manipulated to yield a final nicotine composition that is a liquid, a solid, a tablet, a pill, a powder, or any other desired form.

Although an embodiment has been described in detail for purposes of illustration, various modifications may be made without departing from the scope and spirit of the invention. Accordingly, the invention is not to be limited, except as by the appended claims.

What is claimed is:

1. A method of producing a consumable nicotine composition, comprising:
measuring a large quantity of nicotine;
diluting the large quantity of nicotine into one or more successive intermediate solutions, a last of which constitutes a final solution;
apportioning the final solution so that each portion contains a precise quantity of nicotine appropriate for consumption in a single use by a single person; and
introducing each portion into its own single-serving beverage.

2. The method of claim 1, wherein each successive intermediate solution has a lower nicotine concentration than that of the solution immediately preceding it.

3. The method of claim 1, wherein a nicotine concentration of between 0.0001% and 0.1% by volume is attained in the beverages after addition of each portion thereto.

4. The method of claim 1, wherein a primary component of the beverage is water.

5. The method of claim 1, wherein a primary component of the beverage is carbonated water.

6. The method of claim 1, wherein a primary component of the beverage is a natural juice.

7. The method of claim 1, wherein the final solution is liquid.

8. The method of claim 1, wherein the final solution is non-liquid.

9. The method of claim 1, wherein the portions are introduced into the beverages prior to sale or distribution.

10. A method of producing a consumable nicotine composition, comprising:
measuring a large quantity of nicotine;
diluting the large quantity of nicotine into one or more successive intermediate solutions, a last of which constitutes a final solution;
apportioning the final solution so that each portion contains a precise quantity of nicotine appropriate for consumption in a single use by a single person; and
packaging each portion in a separate single-use dispenser.

11. The method of claim 10, wherein the single-use dispensers are sold or distributed to be introduced into beverages by consumers.

12. The method of claim 10, wherein the single-use dispenser is an eyedropper.

13. The method of claim 10, wherein the single-use dispenser is a porous or semi-porous pouch, similar to a tea bag.

14. The method of claim 10, wherein the single-use dispenser is a packet.

15. The method of claim 10, wherein the single-use dispenser is a capped tube.

16. The method of claim 10, wherein the single-use dispenser is a dissolvable pill or tablet.

17. The method of claim 10, wherein a nicotine concentration of between 0.0001% and 0.1% by volume is attained in the beverage after adding the contents of the single-use dispenser thereto.
